SEQUENCE LISTING (6 sequences)

SEQ ID NO: 1 - protein, 374 aa, Homo sapiens
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 2 - protein, 374 aa, Artificial Sequence
  Synthetic construct
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 3 - protein, 374 aa, Artificial Sequence
  Synthetic construct
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPYPGCRHGTCSTPWQCLCDEGWGGLYCDQDLNYCTHHSPCKNGATCHNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 4 - protein, 374 aa, Artificial Sequence
  Synthetic construct
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHPGCRHGTCSTPWQCLCDEGWGGLYCDQDLNYCTHHSPCKNGATCRNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 5 - protein, 374 aa, Artificial Sequence
  Synthetic construct
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHPGCRHGTCSTPWQCLCDEGWGGLYCDQDLNYCTHHSPCKNGATCRNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

SEQ ID NO: 6 - protein, 374 aa, Artificial Sequence
  Synthetic construct
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPYPGCRHGTCSTPWQCLCDEGWGGLYCDQDLNYCTHHSPCKNGATCHNSGPRSYTCTCRPGYTGVDCELELSECDSNPCRNGGSCKDQEDGYHCLCPPGYYGLHCEHSTLSCADSPCFNGGSCRERNQGANYACECPPNFTGSNCE

(19) United States
(12) Patent Application Publication
(10) Pub. No.: US 2022/0241335 A1
(43) Pub. Date: Aug. 4, 2022

(54) ENGINEERING NOTCH LIGANDS TO ENHANCE THE ANTI-TUMOR ACTIVITY OF ADOPTIVELY TRANSFERRED T CELLS

(71) Applicant: H. LEE MOFFITT CANCER CENTER AND RESEARCH INST5ITUTE, INC., Tampa, FL (US)

(72) Inventors: Vincent LUCA, Tampa, FL (US); Paulo RODRIGUEZ, Tampa, FL (US); David GONZALEZ-PEREZ, Tampa, FL (US)

(21) Appl. No.: 17/626,315

(22) PCT Filed: Jul. 13, 2020
(86) PCT No.: PCT/US2020/041765

§ 371 Date: Jan. 11, 2022

Related U.S. Application Data
(60) Provisional application No. 62/872,976, filed on Jul. 11, 2019.

(51) Int. Cl. A61K 35/17; A61P 35/00; C12N 5/0783; A61K 38/17

(52) U.S. Cl. CPC A61K 35/17; A61K 38/1774; C12N 5/0638; A61P 35/00

(57) ABSTRACT

Disclosed are compositions and methods for engineered Notch ligands for activating Notch signaling, enhancing eh efficacy of adoptive T cell immunotherapy, making a T cell resistant to tumor suppression, and/or treating a cancer. In one aspect, disclosed herein are methods of activating Notch signaling comprising contacting T cells (such as, for example CD8+ T cells, CD4+ T cells, chimeric antigen receptor 15 (CAR) T cells, tumor infiltrating lymphocytes (TILs), and/or marrow infiltrating lymphocytes (MILS)) with a chemically designed Notch ligand.

This invention was made with government support under Grant No. CA204738 awarded by the National Institutes of Health. The government has certain rights in the invention.

### I. BACKGROUND

1. The immunosuppressive effect induced by the tumor microenvironment represents a major obstacle for the success of promising T cell-based immunotherapies, including tumor-expanded T cells, chimeric antigen receptors (CAR)-T cells, and chimeric endocrine receptor (CER)-T cells. What are needed are new strategies that render T cells able to display their programmed anti-tumor capacity after adoptive transfer.

### II. SUMMARY

2. Disclosed are methods and compositions related to engineered Notch ligands.

3. In one aspect, disclosed herein are methods of activating Notch signaling comprising contacting T cells (such as, for example CD8+ T cells, CD4+ T cells, chimeric antigen receptor (CAR) T cells, tumor infiltrating lymphocytes (TILs), and/or marrow infiltrating lymphocytes (MILS)) with a chemically designed Notch ligand; wherein the chemically designed Notch ligand comprises an engineered DLL4 protein comprising a conservative amino acid substitution at a residue corresponding to residues 28, 107, 143, 194, and 206 as set forth in SEQ ID NO: 1 and further comprising at least one conservative amino acid substitution at residues 256 , 257, 271, 280, 301, and 305 as set forth in SEQ ID NO: 1.

4. Also disclosed herein are methods of enhancing the efficacy of adoptive T cell immunotherapy (such as, for example immunotherapy employing CAR T cells, TILs, and/or MILs) comprising contacting T cells (such as, for example CD8+ T cells, CD4+ T cells, chimeric antigen receptor (CAR) T cells, tumor infiltrating lymphocytes (TILs), and/or marrow infiltrating lymphocytes (MILS)) with a chemically designed Notch ligand; wherein the chemically designed Notch ligand comprises an engineered DLL4 protein comprising a conservative amino acid substitution at a residue corresponding to residues 28, 107, 143, 194, and 206 as set forth in SEQ ID NO: 1 and further comprising at least one conservative amino acid substitution at residues 256 , 257, 271, 280, 301, and 305 as set forth in SEQ ID NO: 1

5. In one aspect, disclosed herein are methods of making a T cell resistant to tumor suppression comprising contacting T cells (such as, for example CD8+ T cells, CD4+ T cells, chimeric antigen receptor (CAR) T cells, tumor infiltrating lymphocytes (TILs), and/or marrow infiltrating lymphocytes (MILS)) with a chemically designed Notch ligand that renders anti-tumor T cells (such as, for example CD8+ T cells, CD4+ T cells, chimeric antigen T cells, tumor infiltrating lymphocytes, and/or marrow infiltrating lymphocytes) refractory to the tumor microenvironment; wherein the chemically designed Notch ligand comprises an engineered DLL4 protein comprising a conservative amino acid substitution at a residue corresponding to residues 28, 107, 143, 194, and 206 as set forth in SEQ ID NO: 1 and further comprising at least one conservative amino acid substitution at residues 256 , 257, 271, 280, 301, and 305 as set forth in SEQ ID NO: 1.

6. In one aspect, disclosed herein are methods of treating a cancer in a subject comprising administering to the subject a T cell that has had its efficacy enhanced by any preceding aspect, been made resistant to the tumor microenvironment by any preceding aspect, and/or had its Notch signaling by activated by any preceding aspect. Also disclosed are methods of treating a cancer in a subject comprising obtaining a T cell (such as, for example CD8+ T cells, CD4+ T cells, chimeric antigen receptor (CAR) T cells, tumor infiltrating lymphocytes (TILs), and/or marrow infiltrating lymphocytes (MILS)); contacting the T cell with a chemically designed Notch ligand; wherein the chemically designed Notch ligand comprises an engineered DLL4 protein comprising a conservative amino acid substitution at a residue corresponding to residues 28, 107, 143, 194, and 206 as set forth in SEQ ID NO: 1 and further comprising at least one conservative amino acid substitution at residues 256 , 257, 271, 280, 301, and 305 as set forth in SEQ ID NO: 1; and administering the T cell to the subject with the cancer.

7. Also disclosed herein are methods of activating Notch signaling, methods of enhancing eh efficacy of adoptive T cell immunotherapy, methods of making a T cell resistant to tumor suppression, and methods of treating a cancer of any preceding aspect; wherein the engineered DLL4 protein comprises a conservative amino acid substitution at a residue corresponding to residues 28 (for example a G28S substitution), 107 (for example a F107L substitution), 143 (for example a I143F substation), 194 (for example a H194Y substation), and 206 (for example a L206P substitution) as set forth in SEQ ID NO: 1 and further comprising at least one conservative amino acid substitution at residues 256 , 257, 271, 280, 301, and 305 as set forth in SEQ ID NO: 1

8. In one aspect, also disclosed herein are methods of activating Notch signaling, methods of enhancing the efficacy of adoptive T cell immunotherapy, methods of making a T cell resistant to tumor suppression and methods of treating a cancer of any preceding aspect; wherein the amino acid at residue 256 of the engineered DLL4 protein comprises a histidine, tyrosine, phenylalanine, leucine, asparagine, isoleucine, valine, or aspartic acid (such as, for example, a H256Y substitution, H256F substitution, H256L substitution, H256N substitution, H256I substitution, H256V substitution, or H256D substitution).

9. Also disclosed herein are methods of activating Notch signaling, methods of enhancing eh efficacy of adoptive T cell immunotherapy, methods of making a T cell resistant to tumor suppression and methods of treating a cancer of any preceding aspect; wherein the amino acid at residue 257 of the engineered DLL4 protein comprises a proline, histidine, leucine, isoleucine, threonine, asparagine, tyrosine, serine, or phenylalanine (such as, for example, a N257P substitution, N257H substitution, N257L substitution, N257I substitution, N257T substitution, N257Y substitution, N257S substitution, or N257F substitution).

10. In one aspect, disclosed herein are methods of activating Notch signaling, methods of enhancing eh efficacy of adoptive T cell immunotherapy, methods of making a T cell resistant to tumor suppression and methods of treating a cancer of any preceding aspect; wherein the amino acid at residue 271 of the engineered DLL4 protein comprises a leucine, proline, histidine, asparagine, threonine, or isoleucine (such as, for example, a T271L substitution, T271P substitution, T271H substitution, T271N substitution, or T271I substitution).

11. Also disclosed herein are methods of activating Notch signaling, methods of enhancing eh efficacy of adoptive T cell immunotherapy, methods of making a T cell resistant to tumor suppression and methods of treating a cancer of any preceding aspect; wherein the amino acid at residue 280 of the engineered DLL4 protein comprises a phenylalanine, leucine, tyrosine, or histidine (F280Y substitution, F280L substitution, or F280H substitution).

12. In one aspect, also disclosed herein are methods of activating Notch signaling, methods of enhancing eh efficacy of adoptive T cell immunotherapy, methods of making a T cell resistant to tumor suppression and methods of treating a cancer of any preceding aspect; wherein the amino acid at residue 301 of the engineered DLL4 protein comprises a serine, asparagine, arginine, or histidine (S301H substitution, S301N substitution, or S301R substitution).

13. Also disclosed herein are methods of activating Notch signaling, methods of enhancing eh efficacy of adoptive T cell immunotherapy, methods of making a T cell resistant to tumor suppression and methods of treating a cancer of any preceding aspect; wherein the amino acid at residue 305 of the engineered DLL4 protein comprises a glutamine, proline, arginine, or leucine (Q305P substitution, Q305R substitution, or Q305L substitution).

14. In one aspect, disclosed herein are methods of activating Notch signaling, methods of enhancing eh efficacy of adoptive T cell immunotherapy, methods of making a T cell resistant to tumor suppression and methods of treating a cancer of any preceding aspect; wherein the engineered DLL4 protein comprises SEQ ID NO: 2, SEQ ID NO: 3, SEQ ID NO: 4, SEQ ID NO: 5, or SEQ ID NO: 6.

15. Also disclosed herein are engineered DLL4 proteins comprising a conservative amino acid substitution at a residue corresponding to residues 28 (for example a G28S substitution), 107 (for example a F107L substitution), 143 (for example a I143F substation), 194 (for example a H194Y substation), and 206 (for example a L206P substitution) as set forth in SEQ ID NO: 1 and/or comprising at least one conservative amino acid substitution at residues 256 , 257, 271, 280, 301, and 305 as set forth in SEQ ID NO: 1

16. In one aspect, disclosed herein are engineered DLL4 proteins of any preceding aspect; wherein the amino acid at residue 256 of the engineered DLL4 protein comprises a histidine, tyrosine, phenylalanine, leucine, asparagine, isoleucine, valine, or aspartic acid (such as, for example, a H256Y substitution, H256F substitution, H256L substitution, H256N substitution, H256I substitution, H256V substitution, or H256D substitution).

17. Also disclosed herein are engineered DLL4 proteins of any preceding aspect; wherein the amino acid at residue 257 of the engineered DLL4 protein comprises a proline, histidine, leucine, isoleucine, threonine, asparagine, tyrosine, serine, or phenylalanine (such as, for example, a N257P substitution, N257H substitution, N257L substitution, N257I substitution, N257T substitution, N257Y substitution, N257S substitution, or N257F substitution).

18. In one aspect, disclosed herein are engineered DLL4 proteins of any preceding aspect; wherein the amino acid at residue 271 of the engineered DLL4 protein comprises a leucine, proline, histidine, asparagine, threonine, or isoleucine (such as, for example, a T271L substitution, T271P substitution, T271H substitution, T271N substitution, or T271I substitution).

19. Also disclosed herein are engineered DLL4 proteins of any preceding aspect; wherein the amino acid at residue 280 of the engineered DLL4 protein comprises a phenylalanine, leucine, tyrosine, or histidine (F280Y substitution, F280L substitution, or F280H substitution).

20. In one aspect, disclosed herein are engineered DLL4 proteins of any preceding aspect; wherein the amino acid at residue 301 of the engineered DLL4 protein comprises a serine, asparagine, arginine, or histidine (S301H substitution, S301N substitution, or S301R substitution).

21. Also disclosed herein are engineered DLL4 proteins of any preceding aspect; wherein the amino acid at residue 305 of the engineered DLL4 protein comprises a glutamine, proline, arginine, or leucine (Q305P substitution, Q305R substitution, or Q305L substitution).

22. In one aspect, disclosed herein are engineered DLL4 proteins of any preceding aspect, wherein the DLL4 protein comprises the sequence as set forth in SEQ ID NO: 2, SEQ ID NO: 3, SEQ ID NO: 4, SEQ ID NO: 5, or SEQ ID NO: 6.

### III. BRIEF DESCRIPTION OF THE DRAWINGS

23. The accompanying drawings, which are incorporated in and constitute a part of this specification, illustrate several embodiments and together with the description illustrate the disclosed compositions and methods.

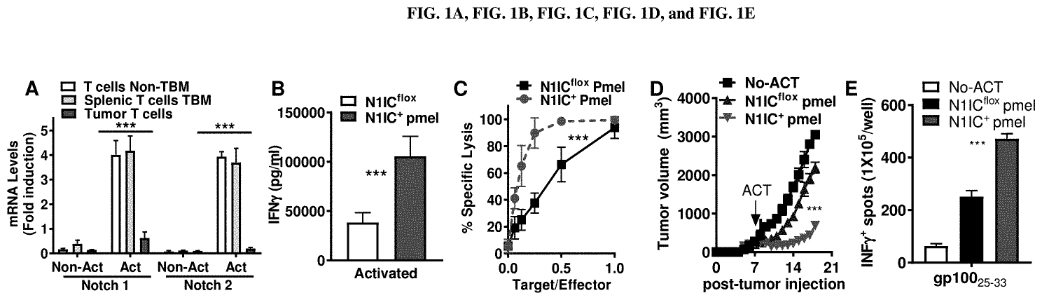

24. FIGS. 1A, 1B, and 1 show the expression of N1IC in CD8+ T cells overcomes MDSC-induced anergy. FIG. 1A shows Notch 1-2 in T cells from tumor and spleens of LLC-bearing (TBM) or naive mice activated with αCD3/28 for 24 hours. FIGS. 1B and 1C show N1IC+ or flox Pmel T cells were primed with the specific peptide for 72 hours and tested for their ability to produce IFNγ and kill [51Cr]-labeled EL4 tumor cells loaded with gp100₂₅₋₃₃. FIG. 1D shows N1IC+ or N1IC− Pmel cells were primed for 48 hours and transferred into mice bearing B16 tumors for 7 days. Tumor volume was then tested. FIG. 1E shows that spleens were taken 10 days later, challenged with KVPRNQDWL, and tested for IFNγ. ***, P<0.001

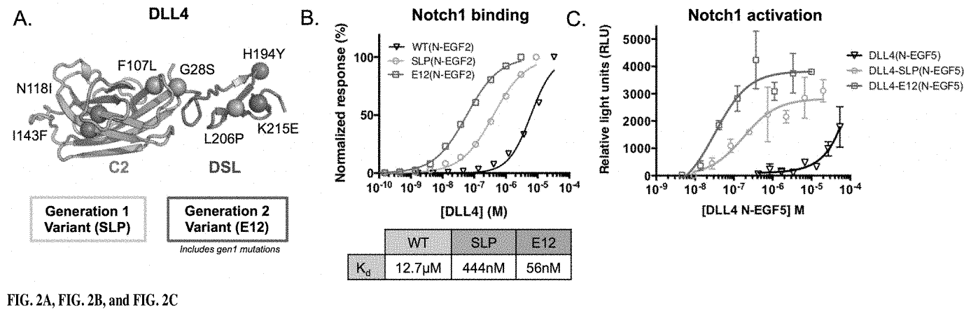

25. FIGS. 2A, 2B, and 2C show evolved DLL4 ligands have enhanced binding and signaling activity. FIG. 2A shows affinity-enhancing mutations in DLL4 SLP and E12 variants. FIG. 2B shows SPR was used to determine the Kd of DLL4 variants relative to WT. FIG. 2C shows DLL4 SLP and DLL4 E12 signal more potently than DLL4 in a Notch luciferase reporter assay.

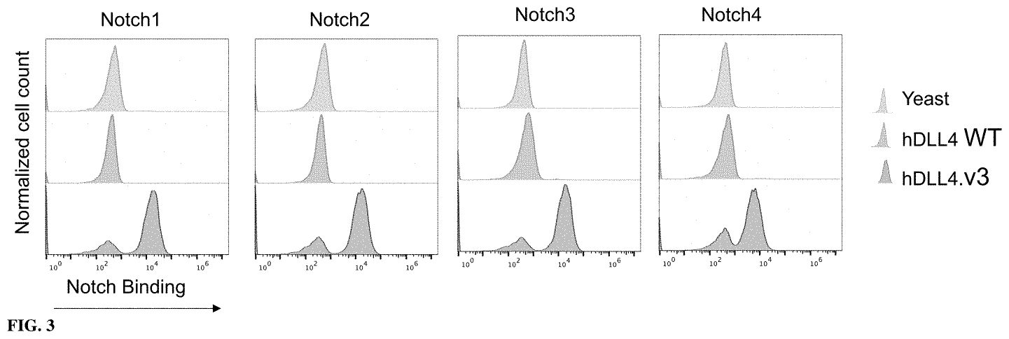

26. FIG. 3 shows the binding of recombinant Notch proteins to yeast-displayed DLL4 ligands.

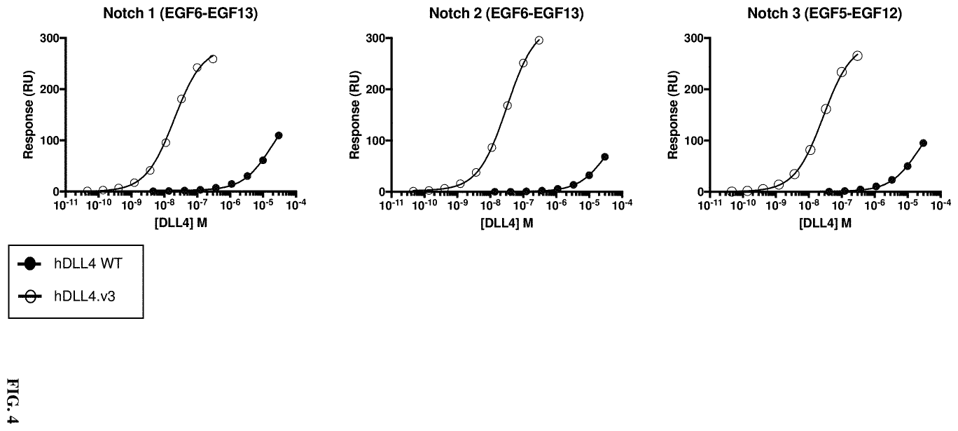

27. FIG. 4 shows the binding affinity of WT DLL4 vs DLL4.v3 variant for Notch 1, Notch2, and Notch 3.

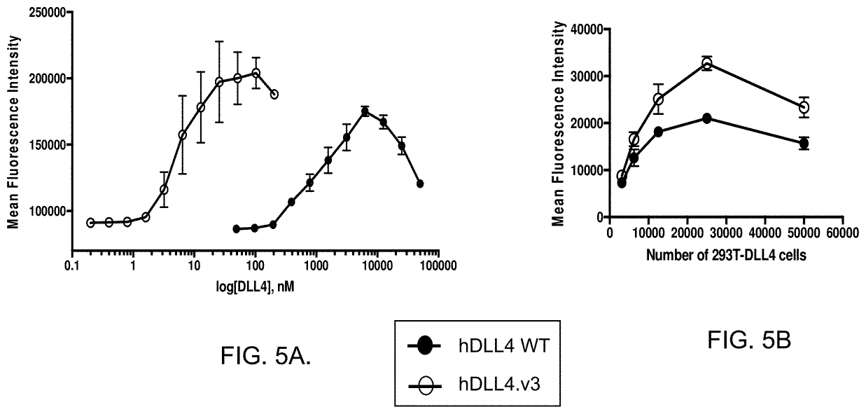

28. FIGS. 5A and 5B show that DLL4.v3 activates Notch more potently than WT DLL4. FIG. 5A shows Notch1 H2B-citrine reporter cells cultured overnight on 96-well plates coated with increasing concentrations of WT DLL4 or DLL4.v3 and Notch1 reporter activity was monitored by flow cytometry. FIG. 5B shows Notch1 H2B-citrine reporter cells were co-cultured overnight with increasing numbers of 293 cells expressing either WT DLL4 or DLL4.v3 and Notch1 reporter activity was monitored by flow cytometry.

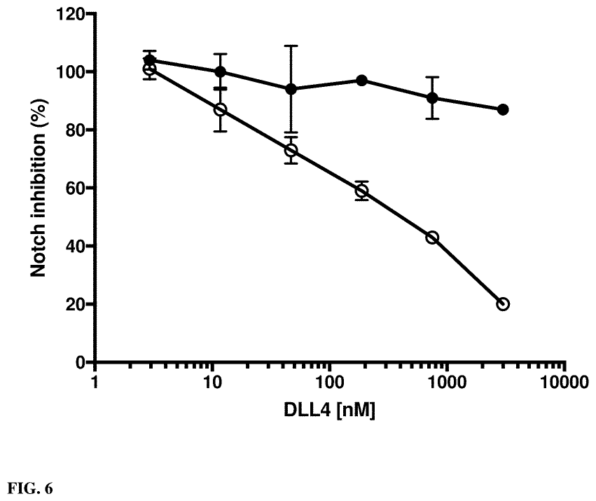

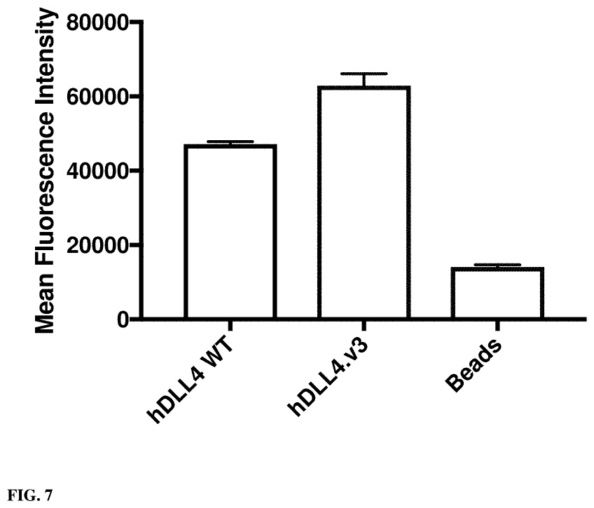

29. FIG. 6 shows that DLL4.v3 inhibits Notch1 activation in a co-culture assay. 30. FIG. 7 shows that magnetic beads coated with DLL4.v3 activate Notch more potently than WT DLL4 beads.

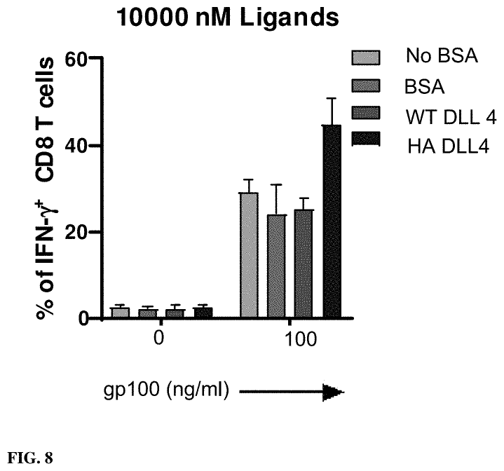

31. FIG. 8 shows IFN-γ secretion by CD8 T cells stimulated with 0 or 100 ng/mL of either wildtype DLL4 (WT-DLL4) or high affinity DLL4 (HA-DLL4) and unstimulated controls with and without bovine serum albumin (BSA).

### IV. DETAILED DESCRIPTION

32. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

33. In this specification and in the claims that follow, reference will be made to a number of terms which shall be defined to have the following meanings:

34. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

35. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

36. “Optional” or “optionally” means that the subsequently described event or circumstance may or may not occur, and that the description includes instances where said event or circumstance occurs and instances where it does not.

37. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

38. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

39. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

40. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

41. “Biocompatible” generally refers to a material and any metabolites or degradation products thereof that are generally non-toxic to the recipient and do not cause significant adverse effects to the subject.

42. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

43. A “control” is an alternative subject or sample used in an experiment for comparison purposes. A control can be “positive” or “negative.”

44. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

45. A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

46. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

47. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

48. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

49. A “binding molecule” or “antigen binding molecule” (e.g., an antibody or antigen-binding fragment thereof) as provided herein refers in its broadest sense to a molecule that specifically binds an antigenic determinant In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

50. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

51. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

52. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

53. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular engineered Notch ligand is disclosed and discussed and a number of modifications that can be made to a number of molecules including the engineered Notch ligand are discussed, specifically contemplated is each and every combination and permutation of engineered Notch ligand and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

54. T cell-based immunotherapies have revolutionized the field of cancer therapeutics and represent a real option for the cure of several malignancies. CD8+ T lymphocytes in particular are primary mediators of adaptive anti-tumor immune responses and have emerged as a promising opportunity for the development of therapeutic approaches against cancer. Despite their potential, the immune inhibitory effect induced by the tumor microenvironment remains a major obstacle for the success of T cell-based therapies. Different components of the tumor microenvironment, including immunosuppressive myeloid subsets (macrophages, dendritic cells, and myeloid-derived suppressor cells), regulatory T cells (Treg), chronic inflammatory cytokines, and mediators related with the metabolism of oxygen, glucose or amino acids, play significant roles in the inhibition of the infiltration, survival, and function of the transferred T cells. Although there is a major emphasis in the field in the development of treatments to block the immunoregulatory activity of major stromal populations, there are no efficient therapeutic strategies to render transferred T cells resistant to the effects of the tumor microenvironment. The development of novel platforms that make T cells refractory to tumors can improve the efficacy of cancer immunotherapy. One approach to improving the efficacy of cancer immunotherapy is through the use of engineered Notch ligands.

55. The Notch family of receptors control a highly conserved pathway that regulates the function, development, and differentiation of many cell types, including immune cells. Mammals have four Notch receptors (Notch1-4) that are bound by five ligands of the Jagged (Jagged1 and 2) and Delta-like (DLL1, 3, and 4) families Binding of Notch receptors to their ligands induces a two-step proteolytic process that is mediated by an ADAM protease, followed by a gamma secretase, which leads to the release and nuclear translocation of the Notch intracellular active domain (NICD). Once there, NICD binds to the recombination signal-binding protein-J (RBP-J) and the mastermind-like (MAML1-3) transcriptional co-activators, driving the expression of multiple genes. Treatment of activated CD8⁺ T cells with Notch-cleavage inhibitors impaired T cell proliferation, cytokine production, and cytotoxicity. Also, inhibited Notch signaling was found in tumor-infiltrating T cells. Notably, expression of Notch1 intracellular active form (N1IC) rendered CD8⁺ T cells refractory to the regulatory effects of tumors and significantly enhanced the effect of T cell-based therapy. Despite the relevance of these results, the permanent promotion of Notch in tumor-specific T cells is limited by the fact that under specific conditions, Notch promotes the development of T cell leukemia. Although the model of overexpression of N1IC in mature primed T cells does not trigger T cell malignancy, alternative approaches to transiently promote Notch activity in tumor-specific T cells exist. Thus, the stimulation of Notch signaling in CD8⁺ T cells by engineered Notch ligand variants can mimic the results using transgenic approaches and increases the efficacy of T cell-based immunotherapy.

56. Notch signal diversity is regulated at the level of both ligands and receptors. On the ligand side, it has been demonstrated that various Jagged and DLL proteins can induce different or even opposing cellular responses in processes ranging from angiogenesis to lymphoid development. In T cells, it has been shown that stimulation with Jagged1, DLL1 and DLL4 have distinct functional outcomes that are associated with intrinsic differences in ligand receptor-binding affinities (Jag1<DLL1<DLL4). The presentation of DLL1 or DLL4 ligands on stromal OP9 cell lines is also a requirement for proper ex vivo differentiation of T cells from hematopoietic stem cells, indicating that secreted co-factors can further potentiate T cell function. Therefore, adoptively transferred CD8+ T cells uniquely respond to different Notch signaling thresholds, which can be identified through stimulation with cell-presented ligands that have been engineered to have a range of different binding affinities.

57. In one aspect, disclosed herein are methods of activating Notch signaling comprising contacting T cells (such as, for example CD8+ T cells, CD4+ T cells, chimeric antigen receptor (CAR) T cells, tumor infiltrating lymphocytes (TILs), and/or marrow infiltrating lymphocytes (MILS)) with a chemically designed Notch ligand; wherein the chemically designed Notch ligand comprises an engineered DLL4 protein.

58. Also disclosed herein are methods of enhancing the efficacy of adoptive T cell immunotherapy (such as, for example immunotherapy employing CAR T cells, TILs, and/or MILs) comprising contacting T cells (such as, for example CD8+ T cells, CD4+ T cells, CAR T cells, TILs, and/or MILs with a chemically designed Notch ligand; wherein the chemically designed Notch ligand comprises an engineered DLL4 protein.

59. In one aspect, disclosed herein are methods of making a T cell resistant to tumor suppression comprising contacting T cells (such as, for example immunotherapy employing CAR T cells, TILs, and/or MILs) with a chemically designed Notch ligand that renders anti-tumor T cells (such as, for example immunotherapy employing CAR T cells, TILs, and/or MILs) refractory to the tumor microenvironment; wherein the chemically designed Notch ligand comprises an engineered DLL4 protein.

60. It is understood and herein contemplated that T cells that have had their efficacy enhanced, been made resistant to the tumor microenvironment, and/or had its Notch signaling by activated by any preceding aspect can be used as an adoptive immunotherapy for the treatment of a cancer. Accordingly, in one aspect, disclosed herein are methods of treating a cancer in a subject comprising administering to the subject a T cell that has had its efficacy enhanced by any of the methods disclosed herein, been made resistant to the tumor microenvironment by any of the methods disclosed herein, and/or had its Notch signaling by any of the methods disclosed herein. For example, disclosed are methods of treating a cancer in a subject comprising obtaining a T cell (such as, for example CD8+ T cells, CD4+ T cells, chimeric antigen receptor (CAR) T cells, tumor infiltrating lymphocytes (TILs), and/or marrow infiltrating lymphocytes (MILS)); contacting the T cell with a chemically designed Notch ligand; wherein the chemically designed Notch ligand comprises an engineered DLL4 protein comprising a conservative amino acid substitution at a residue corresponding to residues 28, 107, 143, 194, and 206 as set forth in SEQ ID NO: 1 and further comprising at least one conservative amino acid substitution at residues 256 , 257, 271, 280, 301, and 305 as set forth in SEQ ID NO: 1; and administering the T cell to the subject with the cancer. The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

61. In one aspect the engineered Notch ligand used in the disclosed methods can comprise engineered DLL4 proteins comprising a conservative amino acid substitution at a residue corresponding to residues 28, 107, 143, 194, and 206 as set forth in SEQ ID NO: 1 and/or comprising or further comprising at least one conservative amino acid substitution at residues 256, 257, 271, 280, 301, and 305 as set forth in SEQ ID NO: 1. For example, the substitution of the engineered DLL4 protein can comprise a glycine to serine substitution at residue 28 (G28S) (as in SEQ ID NOs: 2, 3, and 4), a phenylalanine to leucine substitution at residue 107 (F107L) (as in SEQ ID NOs: 2, 3, and 4), an isoleucine to phenylalanine substitution at residue 143 (I143F) (as in SEQ ID NOs: 2, 3, and 4), a histidine to tyrosine substitution at residue 194 (H194Y) (as in SEQ ID NOs: 2, 3, and 4), and a leucine to proline substitution at residue 206 (L206P) (as in SEQ ID NOs: 2, 3, and 4).

62. As noted above, the engineered DLL4 proteins can comprise one, two , three, four, five, or six substitutions at residues 256, 257, 271, 280, 301, and 305 as set forth in SEQ ID NO: 1. Thus, it is understood and herein contemplated that any one, or combination of any of the residues 256, 257, 271, 280, 301, and 305 can comprise a native residue or substitution. Accordingly, in one aspect, disclosed herein are engineered DLL4 proteins wherein the amino acid at residue 256 comprises a histidine, tyrosine, phenylalanine, leucine, asparagine, isoleucine, valine, or aspartic acid. For example, the amino acid at residue 256 can comprise a histidine or a substitution from histidine in wild-type (WT) human DLL4 (as set forth in SEQ ID NO: 1) to a tyrosine (a H256Y substitution), to a phenylalanine (a H256F substitution), a leucine (a H256L substitution), an asparagine (a H256N substitution), a isoleucine (a H256I substitution), a valine (a H256V substitution), or aspartic acid (a H256D substitution) as set forth in SEQ ID NO: 3 and SEQ ID NO: 6. Similarly, the engineered DLL4 proteins can comprise a proline, histidine, leucine, isoleucine, threonine, asparagine, tyrosine, serine, or phenylalanine at residue 257. Thus, in one aspect, the engineered DLL4 protein can comprise an asparagine at residue 257 or substitution from the asparagine in wild-type (WT) human DLL4 (as set forth in SEQ ID NO: 1) to a tyrosine (a H257Y substitution), a proline (a N257P substitution), a histidine (a N257H substitution), a leucine (a N257L substitution), an isoleucine (a N257I substitution), a threonine (a N257T substitution), a serine (a N257S substitution), or a phenylalanine (a N257F substitution) as set forth in SEQ ID NO: 3, SEQ ID NO: 4, SEQ ID NO: 5, and SEQ ID NO: 6. In one aspect, disclosed herein are engineered DLL4 proteins wherein the amino acid at residue 271 comprises a leucine, proline, histidine, asparagine, threonine, or isoleucine (such as, for example, a wild-type residue as set forth in SEQ ID NO: 1 (i.e., the threonine) or a substitution of the threonine for a leucine (a T271L substitution), a threonine to proline substitution (a T271P substitution), a threonine to histidine substitution (a T271H substitution), a threonine to arginine substitution (a T271N substitution), or a threonine to isoleucine substitution (a T271I substitution) as set forth in SEQ ID NO: 3, SEQ ID NO: 4, SEQ ID NO: 5, and SEQ ID NO: 6. Also disclosed herein are engineered DLL4 proteins, wherein the amino acid at residue 280 comprises a phenylalanine or a substitution of the phenylalanine with a leucine (a F280L substitution), a tyrosine (a F280Y substitution), or histidine (a F280H substitution) as set forth in SEQ ID NO: 3, SEQ ID NO: 4, SEQ ID NO: 5, and SEQ ID NO: 6. Additionally, in one aspect, the disclosed engineered DLL4 proteins can comprise the native serine amino acid at residue 301 as set forth in SEQ ID NO: 1 or comprise a substitution of the serine for an asparagine (a S301N substitution), arginine (a S301R substitution) as set forth in SEQ ID NO: 4 or SEQ ID NO:5, or a histidine (a S301H substitution) as set forth in SEQ ID NO: 4 or SEQ ID NO: 6. Also disclosed herein are engineered DLL4 proteins, wherein the amino acid at residue 305 comprises a glutamine, proline, arginine, or leucine and thus can comprise the wild-type amino acid as set forth in SEQ ID NO: 1 (i.e., a glutamine) or a substitution of the glutamine for a proline (a Q305P substitution), a substitution of the glutamine for an arginine (a Q305R substitution), or a substitution of the glutamine for a leucine (a Q305L substitution) as set forth in SEQ ID NO: 3, SEQ ID NO: 4, SEQ ID NO: 5, and SEQ ID NO: 6. In one aspect, disclosed herein are engineered DLL4 proteins of any preceding aspect, wherein DLL4 protein comprises SEQ ID NO: 2, SEQ ID NO: 3, SEQ ID NO: 4, SEQ ID NO: 5, or SEQ ID NO: 6.

### 1. Homology/Identity

63. It is understood that one way to define any known variants and derivatives or those that might arise, of the disclosed genes and proteins herein is through defining the variants and derivatives in terms of homology to specific known sequences. For example, SEQ ID NO: 1 sets forth a particular sequence of a wild-type human DLL4 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

64. Another way of calculating homology can be performed by published algorithms Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

65. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Peptides

a) Protein Variants

66. As discussed herein there are numerous variants of the DLL4 protein are known and herein contemplated. In addition, to the known functional DLL4 strain variants there are derivatives of the DLL4 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | L |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn;Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

67. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

68. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

69. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

70. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

71. It is understood that one way to define the variants and derivatives of the disclosed proteins herein is through defining the variants and derivatives in terms of homology/identity to specific known sequences. For example, SEQ ID NO: 1 sets forth a particular sequence of human wild-type DLL4 and SEQ ID NOs: 2, 3, 4, 5, and 6 sets forth a particular sequence of a engineered DLL4 proteins. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

72. Another way of calculating homology can be performed by published algorithms Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

73. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

74. It is understood that the description of conservative mutations and homology can be combined together in any combination, such as embodiments that have at least 70% homology to a particular sequence wherein the variants are conservative mutations.

75. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

76. It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

77. Molecules can be produced that resemble peptides, but which are not connected via a natural peptide linkage. For example, linkages for amino acids or amino acid analogs can include CH₂NH—, —CH₂S—, —CH₂—CH₂, —CH═CH— (cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂−S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

78 Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

79. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 3. Pharmaceutical Carriers/Delivery of Pharmaceutical Products

80. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

81. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

82. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

83. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

84. The compositions, including proteins and antibodies, can be used therapeutically in combination with a pharmaceutically acceptable carrier.

85. Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the protein or antibody, which matrices are in the form of shaped articles, e.g., films, liposomes or microparticles. It will be apparent to those persons skilled in the art that certain carriers may be more preferable depending upon, for instance, the route of administration and concentration of composition being administered.

86. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

87. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

88. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

89. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

90. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

91. Compositions for oral administration include powders or granules, suspensions or solutions in water or non-aqueous media, capsules, sachets, or tablets. Thickeners, flavorings, diluents, emulsifiers, dispersing aids or binders may be desirable.

92. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

93. Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

94. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Notch1 in CD8 T Cells Overcomes Tumor-Induced Tolerance and Enhances T Cell-Based Immunotherapy.

95. Herein is shown the induction of Notch1 and 2 in primed T cells. The expression of Notch1 and 2 was compared in T cells from tumors and spleens of Lewis lung carcinoma (LLC)-bearing mice and spleens of mice without tumors. Upregulation of Notch1 and 2 was impaired in tumor-infiltrating T cells compared to those from spleens (FIG. 1A), indicating the regulatory effect of the tumor microenvironment in the expression of Notch in T cells. To further study the role of Notch on CD8⁺ T cells, a strain of mice in which N1IC was conditionally expressed in primed Pmel CD8⁺ T cells that recognize melanoma antigen qp100₂₅₋₃₃ was created. N1IC⁺ pmel mice were developed by crossing pmel, N1IC^{flox}, and granzyme B-driven Cre-recombinase mice. Activation N1IC⁺ pmel CD8⁺ T cells resulted in an increased production of IFNγ and a higher anti-tumor activity (FIG. 1B-C). Next, the increased Notch1 signaling was tested in CD8⁺ T cells overcame the tolerogenic effect induced by tumors. To this end, N1IC⁺ pmel and N1IC^{flox} pmel control T cells were pre-activated in vitro and transferred into mice bearing B16-gp100 tumors for 7 days. Elevated anti-tumor effect and higher frequency of IFNγ-producing CD8⁺ T cells were noted after transfer of primed N1IC⁺ pmel T cells, compared to that induced by N1IC^{flox} pmel T cells (FIG. 1D-E), showing the beneficial effect of promoting Notch1 in CD8⁺ T cells as a strategy to overcome tumor-linked T cell tolerance and to boost the efficacy of T cell-based immunotherapy.

96. Recombinant DLL and Jag ligands bind Notch receptors with intrinsically low affinities (Kd≥10 μM), which limit their utility as tools for biomedical and therapeutic applications. Yeast was used display to engineer affinity-enhancing mutations in rat DLL4 as a means to enhance ligand potency and to stabilize Notch1-DLL4 complexes for co-crystal structure determination. The first generation variant, DLL4 SLP, contained 3 mutations and bound Notch1 with 29-fold higher affinity (444 nM Kd) than wild type DLL4 (12.7 μM Kd), and the second generation variant, DLL4 E12, contained 7 mutations and bound Notch1 with 226-fold higher affinity (56 nM Kd) than wild type DLL4 (FIG. 2A-B). Both the DLL4 SLP and DLL4 E12 variants induced greater maximal signaling than DLL4 in a human Notch1 reporter assay (FIG. 2C), and the variants also exhibited substantially increased potencies in accordance with their augmented binding affinities (FIG. 2B). Therefore, this functional characterization of affinity-matured DLL4 variants highlights the ligand engineering as a strategy to optimize Notch activation levels in T cells for immunotherapy.

### 2. Example 2: Evolve High-Affinity Notch Ligands with a Broad Spectrum of Signal Activation Profiles

97. The data herein describes the development of high-affinity DLL4 ligands with enhanced potency and efficacy in Notch signaling assays (FIG. 2B-C). Because Notch ligands (e.g. DLL1, DLL4, Jag1) with different binding affinities induce distinct phenotypic effects during T cell development, affinity-matured ligands can maximize T cell function through the precise optimization of Notch signaling levels. This can be tested by first evolving ultra-potent ‘third-generation’ DLL4 ligands that bind to Notch1 receptors with even higher affinity. These ligands, along with wild type DLL4 and first- and second-generation ligands, can then be evaluated for their ability to stimulate Notch activation in plate-bound or cell-presented formats. Collectively, the panel of engineered DLL4 proteins provides a comprehensive “toolbox” that enables the precise regulation of Notch levels in CD8+ T cells as well as other Notch-responsive immune cells.

a) Engineer and Purify Human DLL4 Proteins with Enhanced Affinity for Notch1.

98. Structure-guided directed evolution was used to introduce affinity-enhancing mutations into the human DLL4 protein. First, a mutant library encoding ˜>10⁶ variants of the receptor-binding domains of DLL4 was expressed on the surface of yeast cells. Mutations were introduced at specific interface hotspots that were identified in the high-resolution crystal structures of Notch-ligand complexes using degenerate codons. A population of high-affinity binders were then enriched from the library over several rounds of selection (MACS, FACS) using progressively decreasing concentrations of the recombinant Notch extracellular domain. Individual clones were sequenced, and the highest affinity variants can be purified in milligram quantities from insect cells using the established Baculovirus expression system.

99. Using flow cytometry third generation DLL4 (DLL4.v3) ligands were measured for their ability to bind recombinant Notch proteins (FIG. 3). Either WT DLL4 or high-affinity DLL4 (DLL4.v3) was displayed on the surface of yeast and stained with 100 nM concentrations of fluoresecently labeled Notch tetramers. In each case, DLL4.v3 bound Notch to a greater extent than wildtype (WT) DLL4.

100. Next, the binding affinity of each individual variant was determined by surface plasmon resonance. Biotinylated Notch1, Notch2, or Notch3 proteins containing the domains indicated above were immobilized on a surface plasmon resonance sensor chip. Increasing concentrations of WT DLL4 or DLL4.v3 were then flowed over the chip and dissociation constants were determined by fitting the data to a 1:1 equilibrium binding model. As shown in FIG. 4 and Table 3, the binding affinity of DLL4.v3 was at least 3 orders of magnitude stronger than wildtype.

TABLE 3
Affinity equilibrium constant (K_{D})

| | hNotch 1 | hNotch 2 | hNotch 3 |
|---|---|---|---|
| DLL4 ligand | (EGF6-EGF13) | (EGF6-EGF13) | (EGF5-EGF12) |
| WT hDLL4 | 1.79 × 10⁻⁵ | 3.57 × 10⁻⁵ | 2.20 × 10⁻⁵ |
| (N-EGF3) | | | |
| 3a.5_E12 | 2.00 × 10⁻⁸ | 3.14 × 10⁻⁸ | 2.73 × 10⁻⁸ |
| (N-EGF3) | | | |

b) Evaluate the Ability of Evolved DLL4 Ligands to Stimulate Notch1 Reporter Activity in Plate-Bound and Cell-Presented Formats.

101. To determine the potency and efficacy of the DLL4 variants, a signaling assay was performed using a published Notch1 YFP reporter CHO cell line. Notch receptor activation requires the application of mechanical tension, and ligands must therefore either be immobilized on plates or presented on the surfaces of cells to robustly initiate signaling. To monitor ligand-mediated signaling in the plate-bound format, reporter cells were cultured on tissue-culture surfaces that have been coated with various concentrations of each DLL4 ligand, and YFP activity was monitored by flow cytometry. As shown in FIG. 5A and FIG. 5B, DLL4.v3 activates Notch more potently that wildtype DLL4.

102. To monitor ligand signaling in the cell-presented format, stable OP9 stromal cell lines or 293T cells expressing each wild type or DLL4 variant or were generated, and the OP9 cells or 293T cells were co-cultured with Notch1 H2B-citrine reporter cells in the presence of increasing concentrations of soluble inhibitor (either WT DLL4 or DLL4.v3). OP9 and 293T cells are known to secrete factors that facilitate T cell development and the co-culture format therefore has distinct advantages relative to plated ligands for in the functionalization of T cells for adoptive transfer. As shown in FIG. 6, fluorescence was monitored by flow cytometry and analysis of the data revealed that DLL4.v3, but not WT DLL4, was an effective inhibitor. To further elucidate the ability of DLL4.v3 to activate Notch, WT DLL4 or DLL4.v3 proteins were attached to streptavidin-coated magnetic beads and incubated with Notch1 H2B-citrine reporter cells overnight. Notch1 reporter cells were then removed from the plates and citrine fluorescence was monitored using flow cytometry (FIG. 7).

### 3. Example 3: Determine the Effect of Affinity Matured Notch Ligands on the Cytotoxic Function and Therapeutic Activity of Tumor Specific CD8⁺ T Cells

103. Data is shown herein that the activation of Notch signaling in CD8⁺ T cells by engineered Notch ligands promotes anti-tumor cytotoxic activity and enhances the efficacy of T cell-based immunotherapy. This was tested in by focusing on the effect of the expansion of tumor-specific CD8⁺ T cells with different Notch ligand variants in the activation of Notch signaling and the expression of major cytotoxic mediators; and elucidating the effect of this strategy in T cell-immunotherapy models through the adoptive transfer of pmel T cells into B16-bearing mice. These results provide a new platform to improve the efficacy of T cell-immunotherapy in tumors.

a) Engineered Notch Ligands Promote Effector Capacity on Tumor-specific CD8⁺ T Cells

104. The effect of the DLL4 variants produced herein on the activation of Notch and the promotion of T cell cytotoxicity. Therefore, gp100₂₅₋₃₃-activated pmel T cells can be cultured in the presence of Notch ligands having low, medium, or high affinity for the Notch receptors. Then, T cells can be monitored for the expression of full-length and cleaved Notch 1 and 2 and the induction of Notch-inducible genes Hes-1, Hey-1, and DTX1. Also, the ability of the stimulated pmel T cells to kill gp10025-33-B16 tumors and the expression of the cytotoxic molecules granzyme B, perforin, and CD107a. Additionally, the effect of the Notch ligands can be tested on the differentiation of CD8+ T cells into central and effector memory subsets, as shown (FIG. 8). Briefly, T cells from pmel mice were cultured in the presence of 100 ng/ml of gp100 peptide in plates that were pre-coated overnight with 1000 nM Bovine Serum Albumin (BSA), Wild type DLL4 (WT-DLL4), and High affinity DLL4 (HA-DLL4). Cells were monitored 72 hours later for the expression of IFNγ by flow cytometry. T cells coated with DLL4.ve3 (high affinity DLL4), showed increased IFNγ expression. Results showing a heightened Notch signaling and the increased effector function and expression of cytotoxic mediators in Notch ligand-exposed T cells show the benefit of expanding T cells with the developed platform. The results can be validated with anti-CD3/CD28 activated human T cells transduced with FSH-CEM.

b) Tumor-Specific T Cells Conditioned with Notch Ligands and T Cell-Based Immunotherapy.

105. Design. The proof of concept data measuring anti-tumor T cell responses against the model-antigen OVA and tumor-antigen gp100 showed that expression of N1IC in T cells increased the effect of T cell-immunotherapy. Expanding tumor-specific CD8⁺ T cells with plate-bound or cells bearing the engineered Notch ligands triggers their therapeutic efficacy after transfer into tumor-bearing mice. Thus, pmel CD90.1⁺CD8⁺ T cells can be expanded in the presence of the recombinant engineered Notch ligands, after which they can be adoptively transferred into CD90.2⁺ mice bearing established gp100₂₅₋₃₃-B16 melanoma tumors. Then, mice can be monitored for tumor growth and lung metastasis. Also, splenic cells can be collected 10 days after the adoptive transfer, challenged with gp100₂₅₋₃₃, and monitored for parameters of CD8⁺ effector responses, including anti-B16 cytotoxicity and expression of IFNγ and granzyme B. Additionally, transferred CD90.1⁺ pmel T cells in spleens and tumors can be monitored for the expression of T memory and exhaustion markers (CD44, CD62, CD122, PD1, TIM3, LAG-3). A higher anti-tumor effect and an increased effector phenotype in the transferred pmel CD8⁺ T cells is likely to be observed in the mice receiving Notch-ligand-conditioned pmel T cells, confirming the beneficial effect of this approach in T cell immunotherapy. To increase the translation of the studies, the T cell transfers can be repeated w/o non-myeloablative irradiation.

106. Altogether, the data herein shows that the novel ligands demonstrated Notch activity with enhanced potency and efficacy. Further, the expression of Notch1 intracellular active domain in CD8⁺ T cells promoted anti-tumor effector responses, conferred resistance to tumor-induced T cell suppression, and augmented the anti-tumor effects of adoptive T cell therapy. Finally, the results showed that the activation of Notch signaling in CD8⁺ T cells by the novel notch ligands promoted higher resistance to tumor microenvironment and higher persistence following adoptive cell transfer, thereby increasing the effect of T cell-based immunotherapy.

### D. REFERENCES

Adler, S. H. et al. Notch signaling augments T cell responsiveness by enhancing CD25 expression. J. Immunol. 171, 2896-2903 (2003).
Backer, R. A. et al. A central role for Notch in effector CD8(+) T cell differentiation. Nat.Immunol. 15, 1143-1151 (2014).
Balkwill, F. & Mantovani, A. Inflammation and cancer: back to Virchow? Lancet 357, 539-545 (2001).
Benedito, R. et al. The Notch Ligands D114 and Jagged1 Have Opposing Effects on Angiogenesis. Cell 137, 1124-1135 (2009).
Bheeshmachar, G. et al. Evidence for a role for notch signaling in the cytokine-dependent survival of activated T cells. J.Immunol. 177, 5041-5050 (2006).
Cho, O. H. et al. Notch regulates cytolytic effector function in CD8+ T cells. J. Immunol. 182, 3380-3389 (2009).
Cordle, J. et al. A conserved face of the Jagged/Serrate DSL domain is involved in Notch trans-activation and cis-inhibition. Nat. Struct. Mol. Biol. 15, 849-857 (2008).
Fletcher, M. et al. 1-Arginine depletion blunts antitumor T-cell responses by inducing myeloid-derived suppressor cells. Cancer Res. 75, 275-283 (2015).
Guruharsha, K. G., Kankel, M. W. & rtavanis-Tsakonas, S. The Notch signalling system: recent insights into the complexity of a conserved pathway. Nat.Rev.Genet. 13, 654-666 (2012).
Guy, C. S. et al. Distinct TCR signaling pathways drive proliferation and cytokine production in T cells. Nat.Immunol. 14, 262-270 (2013).
Intlekofer, A. M. et al. Effector and memory CD8+ T cell fate coupled by T-bet and eomesodermin. Nat.Immunol. 6, 1236-1244 (2005).
Jaleco, A. C. et al. Differential Effects of Notch Ligands Delta-1 and Jagged-1 in Human Lymphoid Differentiation. J. Exp. Med. 194, 991-1002 (2001).
Joshi, I. et al. Notch signaling mediates G1/S cell-cycle progression in T cells via cyclin D3 and its dependent kinases. Blood 113, 1689-1698 (2009).
Kuijk, L. M. et al. Notch controls generation and function of human effector CD8+ T cells. Blood 121, 2638-2646 (2013).
Lehar, S. M., Dooley, J., Farr, A. G. & Bevan, M. J. Notch ligands Delta 1 and Jagged1 transmit distinct signals to T-cell precursors. Blood 105, 1440-1447 (2005).
Luca, V. C. et al. Structural biology. Structural basis for Notch1 engagement of Delta-like 4. Science 347, 847-853 (2015).
Maekawa, Y. et al. Notch2 integrates signaling by the transcription factors RBP-J and CREB1 to promote T cell cytotoxicity. Nat.Immunol. 9, 1140-1147 (2008).
Maus, M. V. et al. Adoptive immunotherapy for cancer or viruses. Annu Rev Immunol 32, 189-225, doi:10.1146/annurev-immunol-032713-120136 (2014).
Mohtashami, M. et al. Direct comparison of D111- and D114-mediated Notch activation levels shows differential lymphomyeloid lineage commitment outcomes. J. Immunol. Baltim. Md. 1950 185, 867-876 (2010).
Palaga, T., Miele, L., Golde, T. E. & Osborne, B. A. TCR-mediated Notch signaling regulates proliferation and IFN-gamma production in peripheral T cells. J.Immunol. 171, 3019-3024 (2003).
Perales-Puchalt, A. et al. Follicle-Stimulating Hormone Receptor Is Expressed by Most Ovarian Cancer Subtypes and Is a Safe and Effective Immunotherapeutic Target. Clin Cancer Res 23, 441-453, doi:10.1158/1078-0432.CCR-16-0492 (2017).
Raber, P. L. et al. T cells conditioned with MDSC show an increased anti-tumor activity after adoptive T cell based immunotherapy. Oncotarget 7, 17565-17578, doi:10.18632/oncotarget.8197 (2016).
Radtke, F., Fasnacht, N. & MacDonald, H. R. Notch signaling in the immune system. Immunity. 32, 14-27 (2010).
Radtke, F., MacDonald, H. R. & Tacchini-Cottier, F. Regulation of innate and adaptive immunity by Notch. Nat.Rev.Immunol. 13, 427-437 (2013).
Rodriguez, P. C. et al. Arginase I in myeloid suppressor cells is induced by COX-2 in lung carcinoma. J.Exp.Med. 202, 931-939 (2005).
Rodriguez, P. C. et al. Arginase I production in the tumor microenvironment by mature myeloid cells inhibits T-cell receptor expression and antigen-specific T-cell responses. Cancer Res. 64, 5839-5849 (2004).
Rodriguez, P. C. et al. Arginase I-producing myeloid-derived suppressor cells in renal cell carcinoma are a subpopulation of activated granulocytes. Cancer Res. 69, 1553-1560 (2009).
Rodriguez, P. C. et al. L-arginine deprivation regulates cyclin D3 mRNA stability in human T cells by controlling HuR expression. J.Immunol. 185, 5198-5204 (2010).
Rodriguez, P. C., Quiceno, D. G. & Ochoa, A. C. L-arginine availability regulates T-lymphocyte cell-cycle progression. Blood 109, 1568-1573 (2007).
Sadelain, M., Riviere, I. & Riddell, S. Therapeutic T cell engineering. Nature 545, 423-431, doi:10.1038/nature22395 (2017).
Schmitt, T. M., Ciofani, M., Petrie, H. T. & Zúñiga-Pflücker, J. C. Maintenance of T Cell Specification and Differentiation Requires Recurrent Notch Receptor-Ligand Interactions. J. Exp. Med. 200, 469-479 (2004).
Sierra, R. A. et al. Anti-Jagged Immunotherapy Inhibits MDSCs and Overcomes Tumor-Induced Tolerance. Cancer Res 77, 5628-5638, doi:10.1158/0008-5472.CAN-17-0357 (2017).
Sierra, R. A. et al. Rescue of notch-1 signaling in antigen-specific CD8+ T cells overcomes tumor-induced T-cell suppression and enhances immunotherapy in cancer. Cancer Immunol.Res. 2, 800-811 (2014).
Sprinzak, D. et al. Cis-interactions between Notch and Delta generate mutually exclusive signalling states. Nature 465, 86-90 (2010).
Thevenot, P. T. et al. The stress-response sensor chop regulates the function and accumulation of myeloid-derived suppressor cells in tumors. Immunity. 41, 389-401 (2014).
Varnum-Finney, B. et al. Immobilization of Notch ligand, Delta-1, is required for induction of notch signaling. J. Cell Sci. 113 Pt 23, 4313-4318 (2000).
Wang, X. & Ha, T. Defining Single Molecular Forces Required to Activate Integrin and Notch Signaling. Science 340, 991-994 (2013).

### E. SEQUENCES

SEQ ID NO: 1 amino acid sequence for wildtype (WT)
human DLL4 starting at residue 27
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPHNGCRHGTCSTPWQCTCDEGW
GGLFCDQDLNYCTHHSPCKNGATCSNSGQRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE
SEQ ID NO: 2 amino acid sequence for E12 human
DLL4 starting at residue 27
SSVFQLQLQEFINERGVLASGRPCEPGCRTFFRVCLKHFQAVVSPGPCTF
GTVSTPVLGTNSFAVRDDSSGGGRNPLQLPLNFTWPGTFSLIIEAWHAPG
DDLRPEALPPDALISKFAIQGSLAVGQNWLLDEQTSTLTRLRYSYRVICS
DNYYGDNCSRLCKKRNDYFGHYVCQPDGNPSCLPGWTGEYCQQPICLSGC
HEQNGYCSKPAECLCRPGWQGRLCNECIPHNGCRHGTCSTPWQCTCDEGW
GGLFCDQDLNYCTHHSPCKNGATCSNSGQRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE
SEQ ID NO: 3 amino acid sequence for DLL4.v3_812
(N-EGF5) N3a.8_E12 human DLL4 starting at residue
27
SSVFQLQLQEFINERGVLASGRPCEPGCRTFFRVCLKHFQAVVSPGPCTF
GTVSTPVLGTNSFAVRDDSSGGGRNPLQLPLNFTWPGTFSLIIEAWHAPG
DDLRPEALPPDALISKFAIQGSLAVGQNWLLDEQTSTLTRLRYSYRVICS
DNYYGDNCSRLCKKRNDYFGHYVCQPDGNPSCLPGWTGEYCQQPICLSGC
HEQNGYCSKPAECLCRPGWQGRLCNECIPYPGCRHGTCSTPWQCLCDEGW
GGLYCDQDLNYCTHHSPCKNGATCHNSGPRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE
SEQ ID NO: 4 amino acid sequence for DLL4.v3
(N-EGF5) N3a.5_E12 human DLL4 starting at residue
27
SSVFQLQLQEFINERGVLASGRPCEPGCRTFFRVCLKHFQAVVSPGPCTF
GTVSTPVLGTNSFAVRDDSSGGGRNPLQLPLNFTWPGTFSLIIEAWHAPG
DDLRPEALPPDALISKFAIQGSLAVGQNWLLDEQTSTLTRLRYSYRVICS
DNYYGDNCSRLCKKRNDYFGHYVCQPDGNPSCLPGWTGEYCQQPICLSGC
HEQNGYCSKPAECLCRPGWQGRLCNECIPHPGCRHGTCSTPWQCLCDEGW
GGLYCDQDLNYCTHHSPCKNGATCRNSGPRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE
SEQ ID NO: 5: amino acid sequence for DLL4_site
2(N-EGF5) starting at residue 27
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPHPGCRHGTCSTPWQCLCDEGW
GGLYCDQDLNYCTHHSPCKNGATCRNSGPRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE
SEQ ID NO: 6: amino acid sequence for
DLL4_site2_812(N-EGF5) starting at residue 27
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPYPGCRHGTCSTPWQCLCDEGW
GGLYCDQDLNYCTHHSPCKNGATCHNSGPRSYTCTCRPGYTGVDCELELS
ECDSNPCRNGGSCKDQEDGYHCLCPPGYYGLHCEHSTLSCADSPCFNGGS
CRERNQGANYACECPPNFTGSNCE

What is claimed is:

1. A method of activating Notch signaling comprising contacting T cells with a chemically designed Notch ligand.

2. A method of enhancing the efficacy of adoptive T cell immunotherapy comprising contacting T cells with a chemically designed Notch ligand.

3. A method of making a T cell resistant to tumor suppression comprising contacting T cells with a chemically designed Notch ligand that renders anti-tumor T cells refractory to the tumor microenvironment.

4. A method of treating a cancer in a subject comprising administering to the subject a T cell that has had its efficacy enhanced by the method of claim 2, been made resistant to the tumor microenvironment by the method of claim 3, and/or had its Notch signaling by activated by the method of claim 1.

5. A method of treating a cancer in a subject comprising obtaining a T cell; contacting the T cell with a chemically designed Notch ligand; and administering the T cell to the subject with the cancer.

6. The method of any of claims 1-5, wherein the T cell comprises a CD8+ T cell, CD4+ T cell, chimeric antigen receptor (CAR) T cell, tumor infiltrating lymphocyte (TIL), and/or marrow infiltrating lymphocyte (MIL).

7. The method of any of claims 1-6, wherein the engineered Notch ligand comprises an engineered DLL4 protein comprising one or more conservative amino acid substitution at a residue corresponding to residues 28, 107, 143, 194, and 206 as set forth in SEQ ID NO: 1 and/or further comprising at least one conservative amino acid substitution at residues 256, 257, 271, 280, 301, or 305 as set forth in SEQ ID NO: 1.

8. The method of claim 7, wherein the substitution at residue 28 comprises a glysine to serine substitution (G28S).

9. The method of claim 7, wherein the substitution at residue 107 comprises a phenylalanine to leucine substitution (F107L).

10. The method of claim 7, wherein the substitution at residue 143 comprises a isoleucine to phenylalanine substitution (I143F).

11. The method of claim 7, wherein the substitution at residue 194 comprises a histidine to tyrosine substitution (H194Y).

12. The method of claim 7, wherein the substitution at residue 206 comprises a leucine to proline substitution (L206P).

13. The method of any of claims 1-6, wherein the engineered Notch ligand comprises an engineered DLL4 protein comprising one or more conservative amino acid substitution at a residue corresponding to residues 256, 257, 271, 280, 301, or 305 as set forth in SEQ ID NO: 1.

14. The method of any of claims 7-13, wherein the amino acid at residue 256 comprises a histidine, tyrosine, phenylalanine, leucine, asparagine, isoleucine, valine, or aspartic acid (H256Y, H256F, H256L, H256N, H256I, H256V, or H256D).

15. The method of any of claims 7-13, wherein the substitution at residue 256 comprises a histidine to tyrosine substitution (H256Y).

16. The method of any of claims 7-13, wherein the amino acid at residue 257 comprises a proline, histidine, leucine, isoleucine, threonine, asparagine, tyrosine, serine, or phenylalanine (N257P, N257H, N257L, N257I, N257T, N257Y, N257S, or N257F).

17. The method of any of claims 7-13, wherein the substitution at residue 257 comprises an asparagine to proline substitution (N257P).

18. The method of any of claims 7-13, wherein the amino acid at residue 271 comprises a leucine, proline, histidine, asparagine, threonine, or isoleucine (T271L, T271P, T271H, T271N, or T271I).

19. The method of any of claims 7-13, wherein the substitution at residue 271 comprises a threonine to leucine substitution (T271L).

20. The method of any of claims 7-13, wherein the amino acid at residue 280 comprises a phenylalanine, leucine, tyrosine, or histidine (F280Y, F280L, or F280H).

21. The method of any of claims 7-13, wherein the substitution at residue 280 comprises a phenylalanine to tyrosine substitution (F280Y).

22. The method of any of claims 7-13, wherein the amino acid at residue 301 comprises a serine, asparagine, arginine, or histidine (S301H, S301N, or S301R).

23. The method of any of claims 7-13, wherein the substitution at residue 301 comprises a serine to histidine substitution (S301H).

24. The method of any of claims 7-13, wherein the substitution at residue 301 comprises a serine to arginine substitution (S301R).

25. The method of any of claims 7-13, wherein the amino acid at residue 305 comprises a glutamine, proline, arginine, or leucine (Q305P, Q305R, or Q305L).

26. The method of any of claims 7-13, wherein the substitution at residue 305 comprises a glutamine to proline substitution (Q305P).

27. The method of any of claims 7-26, wherein DLL4 protein comprises SEQ ID NO: 3.

28. The method of any of claims 7-26, wherein DLL4 protein comprises SEQ ID NO: 4.

29. The method of any of claims 13-26, wherein DLL4 protein comprises SEQ ID NO: 5.

30. The method of any of claims 13-26, wherein DLL4 protein comprises SEQ ID NO: 6.

31. An engineered DLL4 protein comprising one or more conservative amino acid substitution at a residue corresponding to residues 28, 107, 143, 194, and 206 as set forth in SEQ ID NO: 1.

32. The engineered DLL4 protein of claim 31, wherein the substitution at residue 28 comprises a glysine to serine substitution (G28S).

33. The engineered DLL4 protein of claim 31, wherein the substitution at residue 107 comprises a phenylalanine to leucine substitution (F107L).

34. The engineered DLL4 protein of claim 31, wherein the substitution at residue 143 comprises a isoleucine to phenylalanine substitution (I143F).

35. The engineered DLL4 protein of claim 31, wherein the substitution at residue 194 comprises a histidine to tyrosine substitution (H194Y).

36. The engineered DLL4 protein of claim 31, wherein the substitution at residue 206 comprises a leucine to proline substitution (L206P).

37. The engineered DLL4 protein of any of claims 31-36, further comprising at least one conservative amino acid substitution at residues 256, 257, 271, 280, 301, or 305 as set forth in SEQ ID NO: 1.

38. An engineered DLL4 protein comprising at least one conservative amino acid substitution at residues 256, 257, 271, 280, 301, or 305 as set forth in SEQ ID NO: 1.

39. The engineered DLL4 protein of claim 37 or 38, wherein the substitution at residue 256 comprises a histidine to tyrosine substitution (H256Y).

40. The engineered DLL4 protein of claim 37 or 38, wherein the amino acid at residue 257 comprises a proline, histidine, leucine, isoleucine, threonine, asparagine, tyrosine, serine, or phenylalanine (N257P, N257H, N257L, N257I, N257T, N257Y, N257S, or N257F).

41. The engineered DLL4 protein of claim 40, wherein the substitution at residue 257 comprises an asparagine to proline substitution (N257P).

42. The engineered DLL4 protein of claim 37 or 38, wherein the amino acid at residue 271 comprises a leucine, proline, histidine, asparagine, threonine, or isoleucine (T271L, T271P, T271H, T271N, or T271I).

43. The engineered DLL4 protein of claim 42, wherein the substitution at residue 271 comprises a threonine to leucine substitution (T271L).

44. The engineered DLL4 protein of claim 37 or 38, wherein the amino acid at residue 280 comprises a phenylalanine, leucine, tyrosine, or histidine (F280Y, F280L, or F280H).

45. The engineered DLL4 protein of claim 44, wherein the substitution at residue 280 comprises a phenylalanine to tyrosine substitution (F280Y).

46. The engineered DLL4 protein of claim 37 or 38, wherein the amino acid at residue 301 comprises a serine, asparagine, arginine, or histidine (S301H, S301N, or S301R).

47. The engineered DLL4 protein of claim 46, wherein the substitution at residue 301 comprises a serine to histidine substitution (S301H).

48. The engineered DLL4 protein of claim 46, wherein the substitution at residue 301 comprises a serine to arginine substitution (S301R).

49. The engineered DLL4 protein of claim 37 or 38, wherein the amino acid at residue 305 comprises a glutamine, proline, arginine, or leucine (Q305P, Q305R, or Q305L).

50. The engineered DLL4 protein of claim 49, wherein the substitution at residue 305 comprises a glutamine to proline substitution (Q305P).

51. The engineered DLL4 protein of any of claims 31-50, wherein DLL4 protein comprises SEQ ID NO: 3.

52. The engineered DLL4 protein of any of claims 31-50, wherein DLL4 protein comprises SEQ ID NO: 4.

53. The engineered DLL4 protein of any of claims 38-50, wherein DLL4 protein comprises SEQ ID NO: 5.

54. The engineered DLL4 protein of any of claims 38-50, wherein DLL4 protein comprises SEQ ID NO: 6.
